(19) United States
(12) Patent Application Publication
(10) Pub. No.: US 2006/0094987 A1
(43) Pub. Date: May 4, 2006

(54) Catheter guidewire loading device and method

(76) Inventors: Wilhelmus Petrus Martinus Maria van Erp, Leek (NL); Donna Sakaguchi, Tokyo (JP)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/978,258

(22) Filed: Oct. 30, 2004

(51) Int. Cl. A61M 25/00

(52) U.S. Cl. 600/585

(57) ABSTRACT

A loading device for facilitating the loading of a guidewire into a lumen defined by a catheter. A catheter may have a shaft extending from a proximal end to a distal end, defining one or more lumens having one or more catheter ports. A guidewire has a flexible wire-like structure extending from a proximal end to a distal end. The guidewire loading device defines a passage having a first and second loading port. A portion of a catheter may be partially inserted into one of the loading ports, such that a catheter port communicates with the loading device passage. Then, one of the guidewire ends may be inserted into the other loading port. The guidewire may be more easily inserted through the loading device passage, into the catheter port, and thus into the catheter lumen.

### BACKGROUND AND SUMMARY OF THE INVENTION

1. Technical Background

The present invention relates to medical devices, and more particularly to a device for facilitating the loading of a guidewire into a lumen defined by a catheter.

2. Discussion

There are many different kinds and types of catheters, including for example balloon catheters, diagnostic catheters, guiding catheters, stent delivery system catheters, injection catheters, gene therapy catheters, electrophysiology catheters, therapeutic drug delivery catheters, ultrasound catheters, laser angioplasty catheters, etc.

Catheters often have a flexible shaft extending between a proximal end and a distal end, and may define one or more tubular passages or “lumens” extending through part or all of the catheter shaft. Such lumens often have one or more openings, referred to as “ports.” When such a lumen is adapted to slidingly receive a guidewire, it is referred to as a “guidewire lumen,” and it will generally have a proximal and distal “guidewire port.” The distal guidewire port is often at or near the catheter shaft distal end.

A hub is often affixed to the catheter shaft proximal end. The hub may serve a variety of functions, including providing a handle for manipulating the catheter, and/or defining proximal port(s) communicating with lumen(s) defined by the catheter shaft. When there is a guidewire lumen, its proximal guidewire port may be located at some point along the sidewall of the catheter shaft, or the proximal guidewire port may be defined by a hub.

A guidewire has a flexible wire-like structure extending from a proximal end to a distal end. The guidewire will usually be of a size selected to fit into and slide within a corresponding guidewire lumen of a catheter.

Some catheters and guidewires are very flexible, particularly near their distal ends, to enhance their ability to follow more convoluted or tortuous anatomy. Some catheters and guidewires are very thin, especially those for use in narrow body passages including the vessels of the heart and brain, and may be on the order of a few millimeters or less. As medical products, catheters and their guidewire lumens as well as the guidewires, become more flexible and/or smaller in size, it may become challenging to insert the guidewire into a catheter's guidewire lumen. Also, some catheters have a distal guidewire port that is narrowed or tapered, which may hamper a physician when threading a guidewire into a catheter's guidewire lumen.

Accordingly, a catheter guidewire loading device may be desirable, to more easily insert a guidewire into a catheter's guidewire lumen.

A loading device for facilitating the loading of a guidewire into a catheter lumen may be provided for use with catheters and guidewires. The loading device defines a passage having a first and second loading port. A portion of a catheter may be partially inserted into one of the loading ports, such that a catheter port communicates with the loading device passage. Then, one of the guidewire ends may be inserted into the other loading port. The guidewire may be more easily inserted through the loading device passage, into the catheter port, and thus into the catheter lumen.

The terms “tube” and “tubular” are used in their broadest sense, to encompass any structure arranged at a radial distance around a longitudinal axis. Accordingly, the terms “tube” and “tubular” include any structure that (i) is cylindrical or not, such as for example an elliptical or polygonal cross-section, or any other regular or irregular cross-section; (ii) has a different or changing cross-section along its length; (iii) is arranged around a straight, curving, bent or discontinuous longitudinal axis; (iv) has an imperforate surface, or a periodic or other perforate, irregular or gapped surface or cross-section; (v) is spaced uniformly or irregularly, including being spaced varying radial distances from the longitudinal axis; or (vi) has any desired combination of length or cross-sectional size.

Any suitable material may be used to make loading devices, including polymers and other materials suitable for use with medical devices.

It is of course possible to build various kinds and designs of guidewire loading devices according to the present invention, in various shapes, by various techniques and of various materials, to obtain the desired features. It should be noted that the present invention also relates to methods for making and using guidewire loading devices, during or in preparation for medical treatment of a patient.

These and various other objects, advantages and features of the invention will become apparent from the following description and claims, when considered in conjunction with the appended drawings. The invention will be explained in greater detail below with reference to the attached drawings of a number of examples of embodiments of the present invention.

### BRIEF DESCRIPTION OF THE DRAWINGS

FIG. 1 is an end elevation view of a guidewire loading device;

FIG. 2 is a side elevation view of the guidewire loading device of FIG. 1;

FIG. 3 is a partial cross-sectional view of a guidewire loading device;

FIG. 4 is a partial cross-sectional view of the guidewire loading device of FIG. 1, along the lines 4-4;

FIG. 5 is a partial cross-sectional view of a guidewire loading device, with portions of a catheter and a guidewire;

FIGS. 6-8 and 10 are longitudinal cross-sectional views of guidewire loading devices;

FIGS. 9 and 11 are transverse cross-sectional views of the guidewire loading devices of FIGS. 8 and 10; and

FIG. 12 is a longitudinal cross-sectional view of a guidewire loading device.

### DETAILED DESCRIPTION OF THE PREFERRED EMBODIMENTS

The following description of the preferred embodiments of the present invention is merely illustrative in nature, and as such it does not limit in any way the present invention, its application, or uses. Numerous modifications may be made by those skilled in the art without departing from the true spirit and scope of the invention.

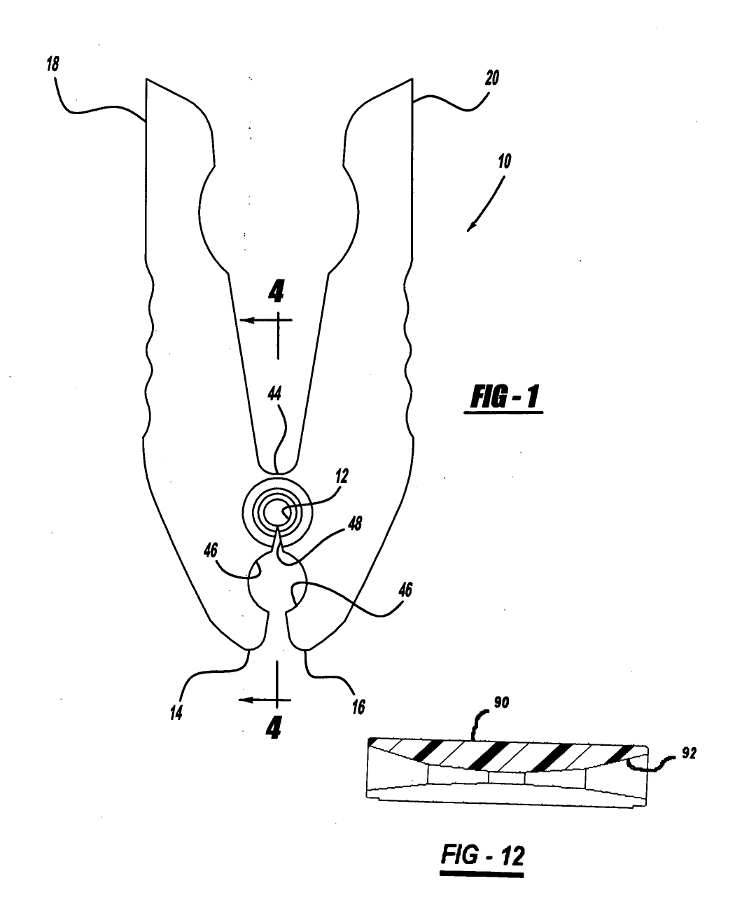

The drawings depict catheter guidewire loading devices along the lines of the present invention. FIGS. 1-5 show catheter guidewire loading devices in an arrangement having an optional additional feature, which is a clip for holding parts of catheter(s) and/or guidewire(s). FIGS. 6-12 show catheter guidewire loading devices alone, without additional features.

A catheter guidewire loading device 10 has a loading passage 12, a first and second clip portion 14 and 16, and a first and second handle portion 18 and 20. Loading passage 12 extends between a first and second end or loading port 22 and 24, has a first and second outer tubular section 26 and 28, a first and second outer tapering section 30 and 32, a first and second inner tubular section 34 and 36, a first and second inner tapering section 38 and 40, and a central tubular section 42.

As shown, the loading passage may be longitudinally symmetrical, so that a physician can insert a catheter into either end of the loading passage. In other words, the loading passage may be designed so as not to require a physician to insert a particular type of medical device into a particular end of the loading passage. Of course, a loading passage of a catheter guidewire loading device may alternately not be symmetrical, designed with each end customized for a particular type of medical device.

A portion of a catheter may be partially inserted into one of the loading ports, such that a catheter port communicates with the loading device passage. Then, one of the guidewire ends may be inserted into the other loading port. The guidewire may be more easily inserted through the loading device passage, into the catheter port, and thus into the catheter lumen.

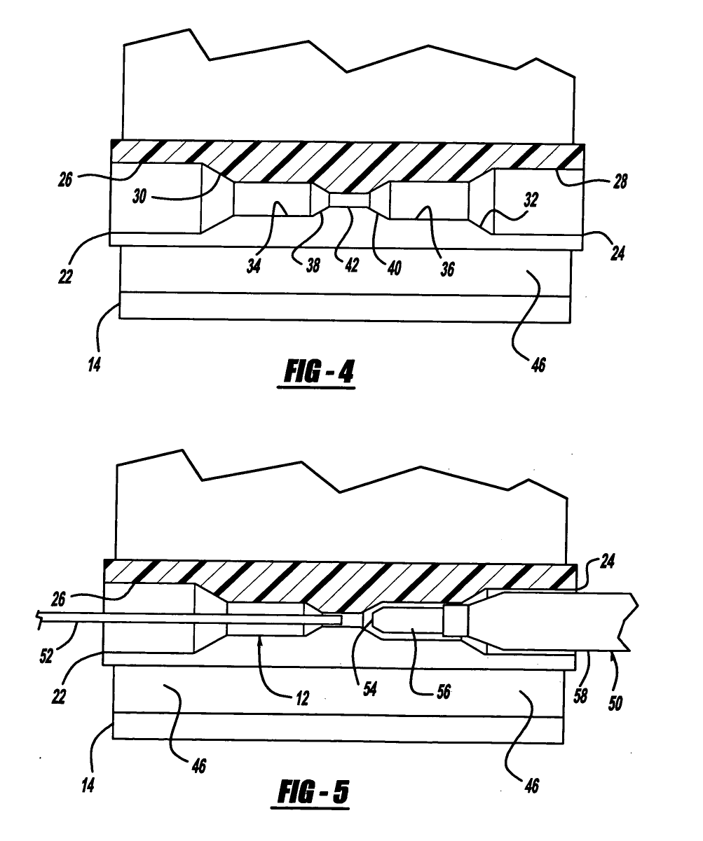

Referring to the drawings, a physician may insert a catheter, such as for example a balloon catheter 50 shown in FIG. 5, into a loading port 24. And the physician may insert a guidewire 52 in through the other loading port 22. Loading passage 12 provides a mutually tapering shape which tends to center both the tip of catheter 50 and the end of guidewire 52. As a result, the end of guidewire 52 is more easily aligned with a guidewire port 54 defined by catheter 50, and inserted through guidewire port 54 into a guidewire lumen defined inside the catheter 50.

Catheter guidewire loading devices may also be designed for use with a variety of different kinds of catheters. Since there are many different types of catheters which have guidewire lumens and guidewire ports, catheter guidewire loading devices may be provided for use with any of them. Examples of different types of catheters include balloon catheters, diagnostic catheters, guiding catheters, stent delivery system catheters, injection catheters, gene therapy catheters, electrophysiology catheters, therapeutic drug delivery catheters, ultrasound catheters, laser angioplasty catheters, etc.

It is also possible to customize a catheter guidewire loading device for specific type(s) of medical devices. For example, a loading passage of a catheter guidewire loading device may be shaped to accommodate specific portions of a catheter and guidewire. A particular example is passage 12 shown in the drawings, in which the tubular sections and tapering sections are arranged to accommodate tubular shaft tip portions 56 and a balloon member 58.

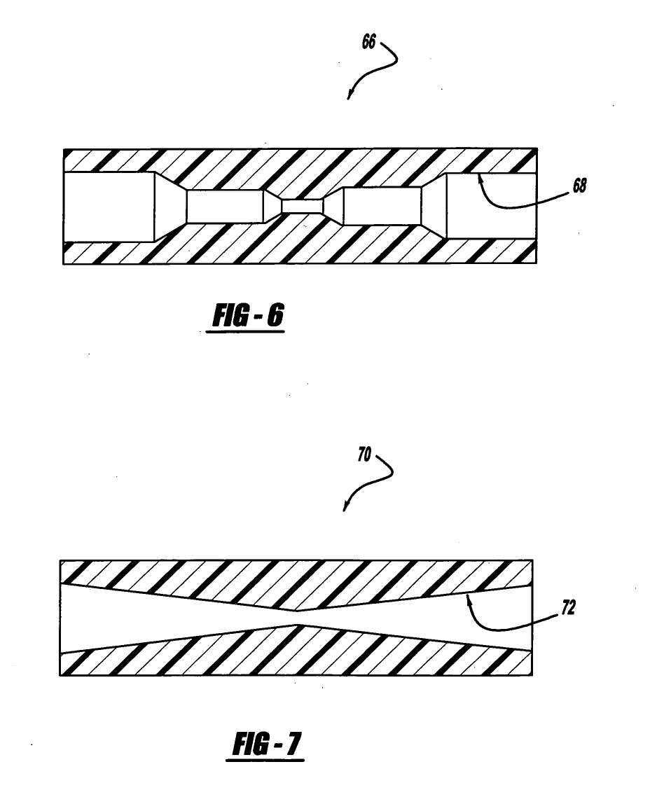

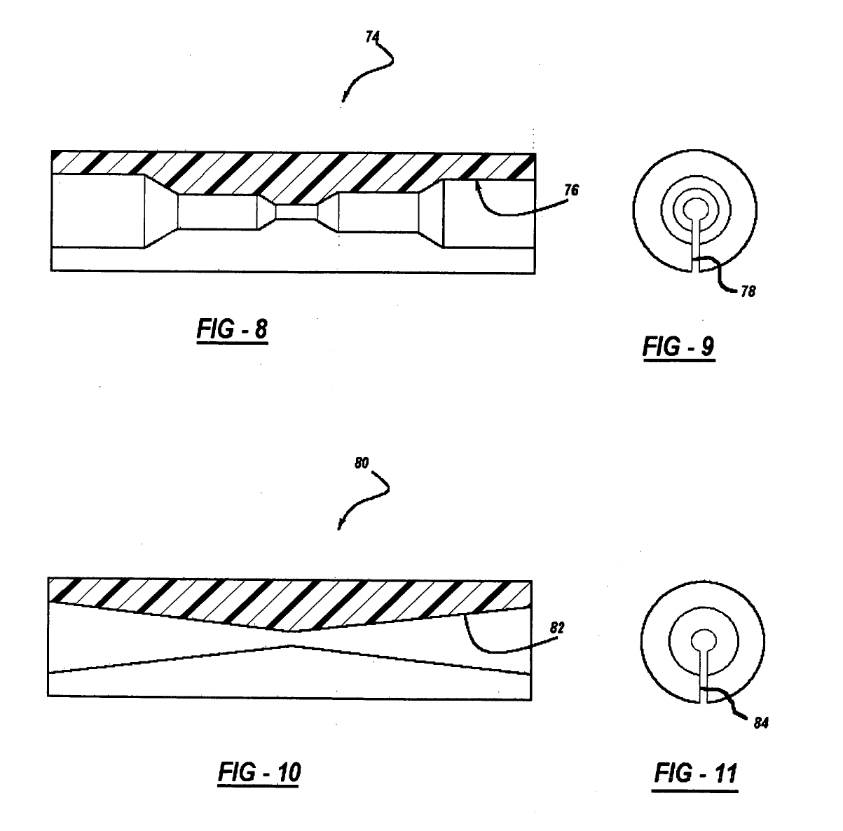

If desired, a loading passage of a catheter guidewire loading device may have various additional shapes, each designed to align the guidewire port of a catheter inserted into one loading port with a guidewire inserted in through another loading port. Other possible shapes for loading passages include a smoothly tapering surface, a venturi shape, or any other suitable shape. For example, FIG. 7 shows a catheter guidewire loading device 70 with loading passage 72 having a double-tapering shape. Another example is shown in FIGS. 8 and 9, in which a catheter guidewire loading device 74 defines a loading passage 76 and a channel 78 for allowing easier removal of a catheter and guidewire from loading device 74. Another example is shown in FIGS. 10 and 11, in which a catheter guidewire loading device 80 defines a loading passage 82 having a double-tapering shape and a channel 84. Yet another example is shown in FIG. 12, in which a catheter guidewire loading device 90 defines a loading passage 92 having a compound tapering shape.

Because the devices shown in FIGS. 1-5 combine features of a resilient clip with a catheter guidewire loading passage, they are combination devices. Handle portions 18 and 20 provide surfaces for holding the catheter guidewire loading device, and also may be squeezed together to actuate the clip features of device 10. A vertex between the handle portions and an upper portion of loading passage 12 cooperate to act as a living hinge 44. Living hinge 44 allows handle portions 18 and 20 to be squeezed together, causing clip portions 14 and 16 to separate, so that a medical device such as a catheter and/or a guidewire may be inserted within a clip passage 46. When squeezing pressure on handle portions 18 and 20 is relieved, the living hinge 44 resiliently causes clip portions 14 and 16 to close toward each other.

Clip portions 14 and 16 are shown in FIG. 1 as being separated in a resting position. Alternately, a catheter guidewire loading device may be provided in which clip portions may be arranged to be in contact with each other in a resting position, to more securely clip onto various medical devices.

The resilient hinge action of living hinge 44 may be made easier by a channel 48 cut through the bottom side of loading passage 12. Such a channel 48 allows the living hinge 44 to operate more freely, so that clip portions 14 and 16 may be used more easily.

Another advantage of channel 48 is that, after device 10 has been used to insert a guidewire 52 into a guidewire port 54 defined by a catheter 58, the device 10 can more easily be removed from the resulting assembly.

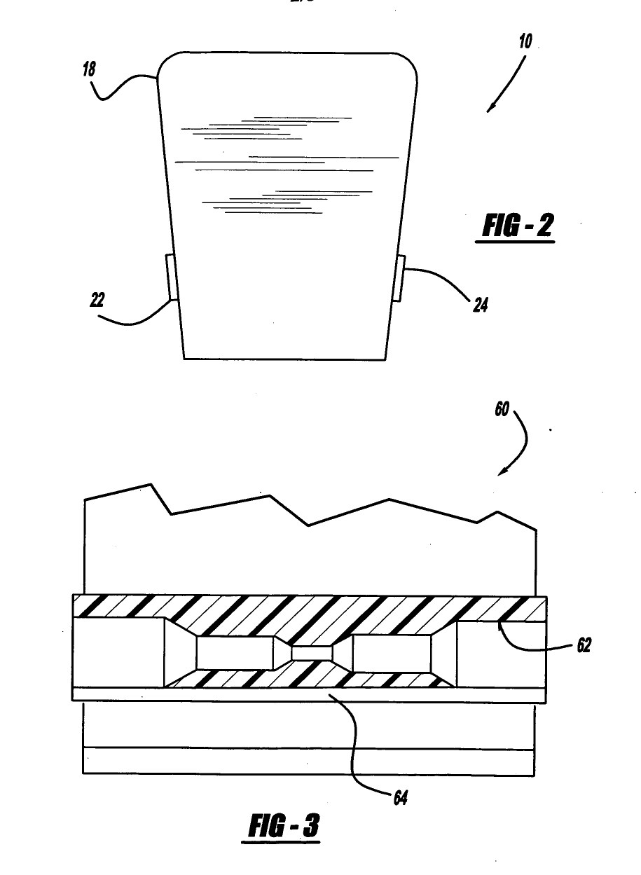

Of course, other arrangements of this type of channel are possible. FIG. 3 shows a catheter guidewire loading device 60 which defines a loading passage 62, and has a partial channel 64. Such a partial channel may provide for greater strength, while still allowing handles or levers to flex a hinge and move clip portions.

Catheter guidewire loading devices are shown in FIGS. 6-12 that are not combination devices. For example, FIG. 6 shows a second catheter guidewire loading device 66 having a simple and elegant design, which defines a loading passage 68. Similarly, FIG. 7 shows catheter guidewire loading device 70 which defines loading passage 72 having a double-tapering shape. FIGS. 8 and 9 show a catheter guidewire loading device 74 defines loading passage 76, as well as channel 78 for allowing easier removal of a catheter and guidewire from loading device 74. FIGS. 10 and 11 shows a catheter guidewire loading device 80 defining loading passage 82 having a double-tapering shape and a channel 84. FIG. 12 shows a catheter guidewire loading device 90 defining a loading passage 92 having a compound tapering shape.

Regarding methods of using catheter guidewire loading devices, various methods may of course be used. For example, a catheter guidewire loading device is provided, defining a loading passage with a pair of loading ports. Second, a catheter tip may be inserted into one of the loading ports of the catheter guidewire loading device. Third, an end of a guidewire may be inserted into the other loading port of the catheter guidewire loading device, with the loading passage tending to center the guidewire and guide the end of the guidewire accurately toward a guidewire port of the catheter. Finally, the guidewire may be further inserted through the catheter guidewire port and into a guidewire lumen defined by the catheter.

Many variations on methods of using a catheter guidewire loading device are possible. For example, a catheter tip and guidewire end may be simultaneously inserted into opposite loading ports of a catheter guidewire loading device.

Another option for methods of using a catheter guidewire loading device is to provide a loading device having a channel, and after using the loading device to insert a guidewire into a guidewire lumen of a catheter, removing the loading device from the resulting catheter and guidewire assembly, by allowing the catheter and guidewire to escape the loading passage laterally through the channel.

Accordingly, catheter guidewire loading device according to the principles of the present invention may be made of any suitable material using a variety of methods. Various polymers have the desired characteristics of strength, resilience, flexibility, biocompatibility and endurance. Many different materials may be used for manufacturing catheter guidewire loading devices of the present invention. For example, various polymers may be used, such as nylons, including Pebax, as well as polycarbonate, polypropelene, ABS, polyethylenes.

It should be understood that an unlimited number of configurations for the present invention could be realized. The foregoing discussion describes merely exemplary embodiments illustrating the principles of the present invention, the scope of which is recited in the following claims. Those skilled in the art will readily recognize from the description, claims, and drawings that numerous changes and modifications can be made without departing from the spirit and scope of the invention.

What is claimed is:

1. A catheter guidewire loading device for loading an end of a guidewire through a guidewire port defined by a catheter, and into a guidewire lumen defined by the catheter and communicating with the guidewire port, comprising:
a tubular component with a sidewall defining a loading passage extending between a first and second loading port, the first and second loading ports having a loading port size, and a central portion of the loading passage having a central size that is smaller than the loading port size; each successive portion of the loading passage proceeding from the central portion toward each of the loading ports having a size that is equal to or smaller than the next outer portion of the loading passage;
such that a distal end of a catheter shaft defining a guidewire port in communication with a guidewire lumen defined by the catheter shaft may be inserted through the first loading port and into the loading passage of the tubular component;
such that an end of a guidewire may be inserted through the second loading port and into the loading passage of the tubular component;
wherein the shape of the loading passage which becomes narrower from the loading ports to the central portion facilitates insertion of the end of the guidewire toward and through the guidewire port defined by the catheter and into the guidewire lumen defined by the catheter.

2. The catheter guidewire loading device of claim 1, wherein the loading passage from the central portion to the first loading port is symmetrical with the loading passage from the central portion to the second loading port.

3. The catheter guidewire loading device of claim 1, wherein each portion of the loading passage has a circular cross-section.

4. The catheter guidewire loading device of claim 1, wherein the loading passage has a smooth surface tapering from each of the loading ports to the central portion.

5. The catheter guidewire loading device of claim 1, wherein the loading passage has a first and second outer tubular section, a first and second outer tapering section, a first and second inner tubular section, a first and second inner tapering section, and a central tubular section; wherein the inner tapering sections are positioned between the center section and the inner tubular sections, and the outer tapering sections are positioned between the inner tubular sections and the outer tubular sections.

6. The catheter guidewire loading device of claim 1, further comprising a channel extending a radial direction outward from the loading passage, and extending the full length of the loading device from the first loading port to the second loading port; wherein an object extending inside the loading passage between the first and second loading ports can pass radially out of the loading passage, through the channel.

7. The catheter guidewire loading device of claim 1, further comprising one or more clip portions and one or more handle portions, and a resilient hinge, such that the handle portion can be actuated to open and close the clip portions, so as to selectively clip onto other devices.

8. A device for loading an end of a guidewire through a guidewire port defined by a catheter, and into a guidewire lumen defined by the catheter and communicating with the guidewire port, and also for selectively clipping the device onto other objects, comprising:
a tubular component with a sidewall defining a loading passage extending between a first and second loading port, the first and second loading ports having a loading port size, and a central portion of the loading passage having a central size that is smaller than the loading port size; each successive portion of the loading passage proceeding from the central portion toward each of the loading ports having a size that is equal to or smaller than the next outer portion of the loading passage;
wherein the loading passage from the central portion to the first loading port is symmetrical with the loading passage from the central portion to the second loading port; and each portion of the loading passage has a circular cross-section;
a channel extending a radial direction outward from the loading passage, and extending the full length of the loading device from the first loading port to the second loading port; wherein an object extending inside the loading passage between the first and second loading ports can pass radially out of the loading passage, through the channel;
such that a distal end of a catheter shaft defining a guidewire port in communication with a guidewire lumen defined by the catheter shaft may be inserted through the first loading port and into the loading passage of the tubular component;
such that an end of a guidewire may be inserted through the second loading port and into the loading passage of the tubular component;
wherein the shape of the loading passage which becomes narrower from the loading ports to the central portion facilitates insertion of the end of the guidewire toward and through the guidewire port defined by the catheter and into the guidewire lumen defined by the catheter; and
clip portions and handle portions affixed to the tubular component, and a resilient hinge, such that the handle portion can be actuated to open and close the clip portions, so as to selectively clip onto other devices.

9. A method of using a catheter guidewire loading device system to facilitate loading an end of a guidewire through a guidewire port defined by a catheter, and into a guidewire lumen defined by the catheter and communicating with the guidewire port, comprising the steps of:
(a) providing a catheter having a shaft defining a guidewire port and a guidewire lumen in communication with the guidewire port;
(b) providing a guidewire having a first and second end;
(c) providing a tubular component with a sidewall defining a loading passage extending between a first and second loading port, the first and second loading ports having a loading port size, and a central portion of the loading passage having a central size that is smaller than the loading port size; each successive portion of the loading passage proceeding from the central portion toward each of the loading ports having a size that is equal to or smaller than the next outer portion of the loading passage;
(d) inserting the catheter shaft through the first loading port and into the loading passage of the tubular component, such that the guidewire port is aligned with the central portion of the loading passage; and inserting an end of the guidewire through the second loading port and into the loading passage of the tubular component;
(e) continuing to insert the guidewire, wherein the shape of the loading passage which becomes narrower from the loading ports to the central portion facilitates insertion of the end of the guidewire toward and through the guidewire port defined by the catheter and into the guidewire lumen defined by the catheter.

10. The method of claim 1, wherein the tubular component further comprises a channel extending a radial direction outward from the loading passage, and extending the full length of the loading device from the first loading port to the second loading port; further comprising the steps following step (e) of: allowing the guidewire to pass radially out of the loading passage, through the channel.
